(19) United States
(12) Patent Application Publication
(10) Pub. No.: US 2019/0282407 A1
(43) Pub. Date: Sep. 19, 2019

(54) THERAPEUTIC SLEEVE

(71) Applicant: Jonathan Raleigh Davis, Washington, UT (US)

(72) Inventor: Jonathan Raleigh Davis, Washington, UT (US)

(21) Appl. No.: 16/352,244

(22) Filed: Mar. 13, 2019

Related U.S. Application Data
(60) Provisional application No. 62/642,452, filed on Mar. 13, 2018.

(51) Int. Cl. A61F 13/08; A61F 7/02; A61F 13/00

(52) U.S. Cl. CPC A61F 13/08; A61F 7/02; A61F 13/00029; A61F 2007/0039; A61F 13/00051; A61F 2007/0238; A61F 13/00034

(57) ABSTRACT

A therapeutic sleeve includes a compression sleeve having an inner sheet and an outer sheet made of flexible fluid impervious material sealingly fixed together to form at least one air pocket therebetween. An inlet valve is fluidly connected to the at least one air pocket to fill the air pocket with air via a pump. The present invention further includes a liner including a sheet having an outer surface and an inner surface. At least one pocket is coupled to the outer surface. A first connector couples the inner surface of the liner to the inner sheet of the compression sleeve. A second connector is configured to removably couple the compression sleeve to itself when wrapped around a limb of an animal.

### CROSS-REFERENCE TO RELATED APPLICATION

This application claims the benefit of priority of U.S. provisional application No. 62/642,452, filed Mar. 13, 2018, the contents of which are herein incorporated by reference.

### BACKGROUND OF THE INVENTION

The present invention relates to therapeutic sleeve and, more particularly, to a compression sleeve with a liner having pockets for cold and hot packs.

### SUMMARY OF THE INVENTION

In one aspect of the present invention, a therapeutic sleeve comprises: a compression sleeve comprising an inner sheet and an outer sheet made of flexible fluid impervious material sealingly fixed together to form at least one air pocket therebetween and an inlet valve fluidly connected to the at least one air pocket; a liner comprising a sheet having an outer surface and an inner surface, wherein at least one pocket is coupled to the outer surface; a first connector configured to removably couple the inner surface of the liner to the inner sheet of the compression sleeve; and a second connector configured to removably couple the compression sleeve to itself when wrapped around a limb of an animal.

In another aspect of the present invention, a method of treating a limb of an animal comprises: providing a compression sleeve comprising an inner sheet and an outer sheet made of flexible fluid impervious material sealingly fixed together to form at least one air pocket therebetween and an inlet valve fluidly connected to the at least one air pocket; providing a liner comprising a sheet having an outer surface and an inner surface, wherein at least one pocket is coupled to the outer surface; placing at least one of a hot pack and a cold pack in the at least one pocket; removably coupling the inner surface of the liner to the inner sheet of the compression sleeve; wrapping the compression sleeve and the liner around the limb of the animal so that the outer surface of the liner is facing towards the limb; removably coupling the compression sleeve to itself, thereby securing the compression sleeve and the liner to the limb; and pumping air into the at least one air pocket through the inlet valve.

In another aspect of the present invention, a therapeutic sleeve comprises: a compression sleeve comprising an inner sheet and an outer sheet made of flexible fluid impervious material sealingly fixed together to form at least one air pocket therebetween and an inlet valve fluidly connected to the at least one air pocket; a liner comprising a sheet having an outer surface and an inner surface, wherein at least one pocket is coupled to the outer surface; at least one of a hot pack and a cold pack removably disposed within the at least one pocket; a first connector coupling the inner surface of the liner to the inner sheet of the compression sleeve; and a second connector configured to removably couple the compression sleeve to itself when wrapped around a limb of an animal.

These and other features, aspects and advantages of the present invention will become better understood with reference to the following drawings, description and claims.

### BRIEF DESCRIPTION OF THE DRAWINGS

FIG. 1 is a front perspective view of an embodiment of the present invention;

FIG. 2 is a rear perspective view of an embodiment of the present invention;

FIG. 3 is a perspective view of an embodiment of the present invention illustrating attachment with a compression sleeve;

FIG. 4 is a perspective view of an embodiment of the present invention illustrating insertion of gel packs;

FIG. 5 is a perspective view of an embodiment of the present invention;

FIG. 6 is a section view of the present invention, taken along lines 6-6 in FIG. 5;

FIG. 7 is a side view of an embodiment of the present invention attached to an animal leg; and

FIG. 8 is a flow chart of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a removable liner with pockets for hot or cold gel packs. The removable liner is removably attached to a pneumatic compression sleeve. The present invention allows the system to be used with or without the removable liner and gel packs as well as targeting certain parts of an injured or sore limb by selectively placing gel packs within certain pockets. The gel packs can be strategically placed only where cold or warm therapy is needed versus over the entire area.

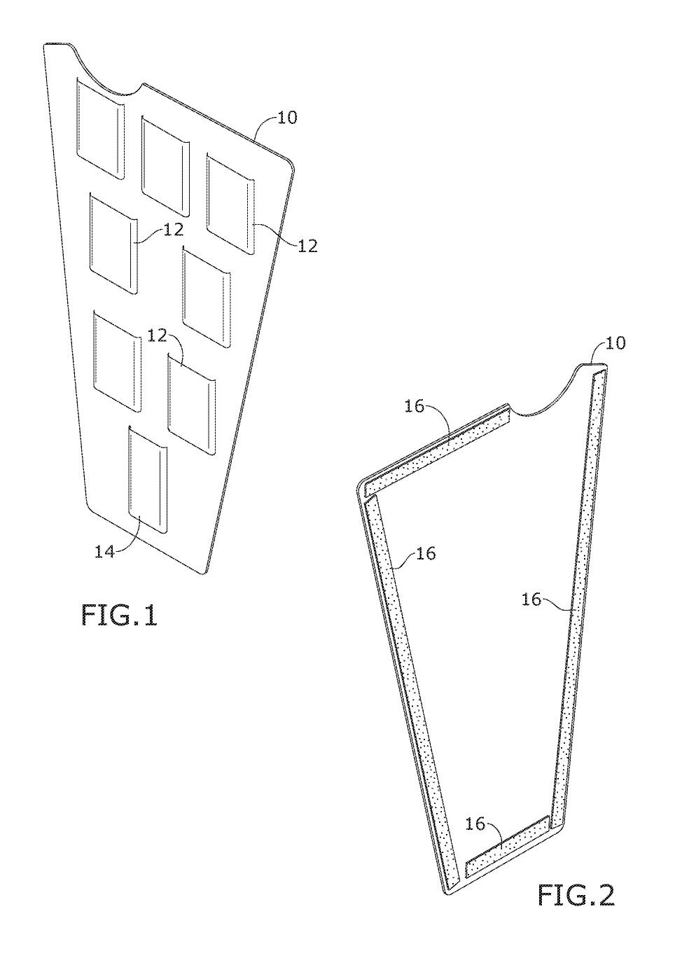

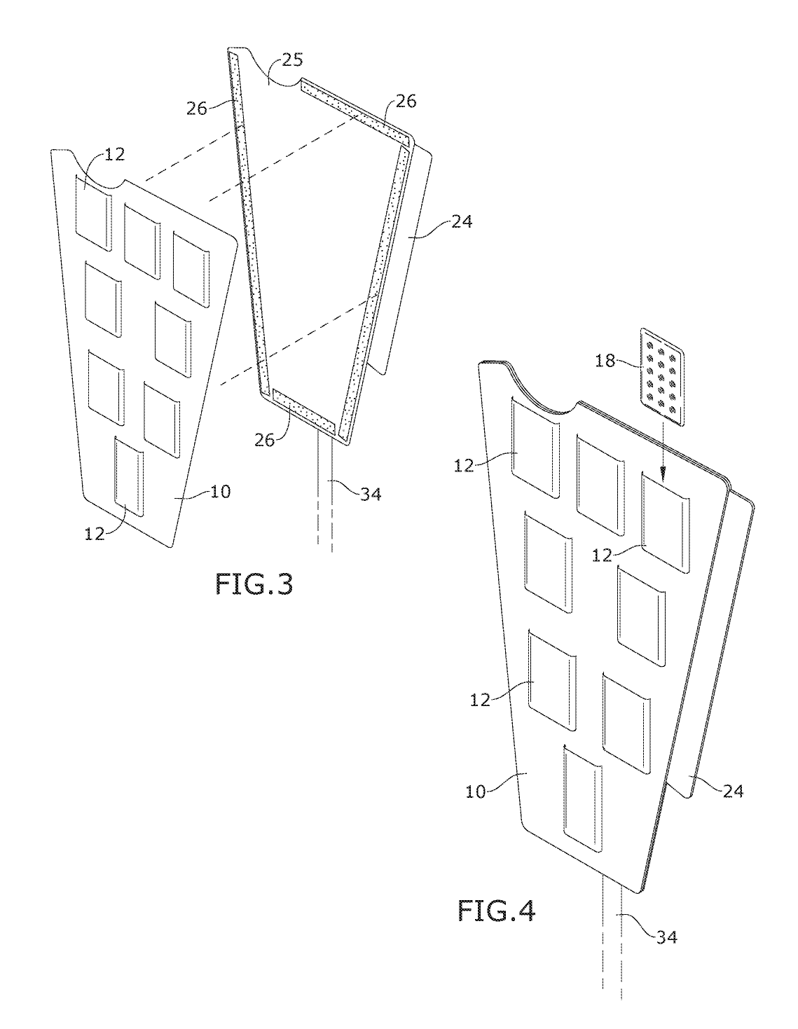

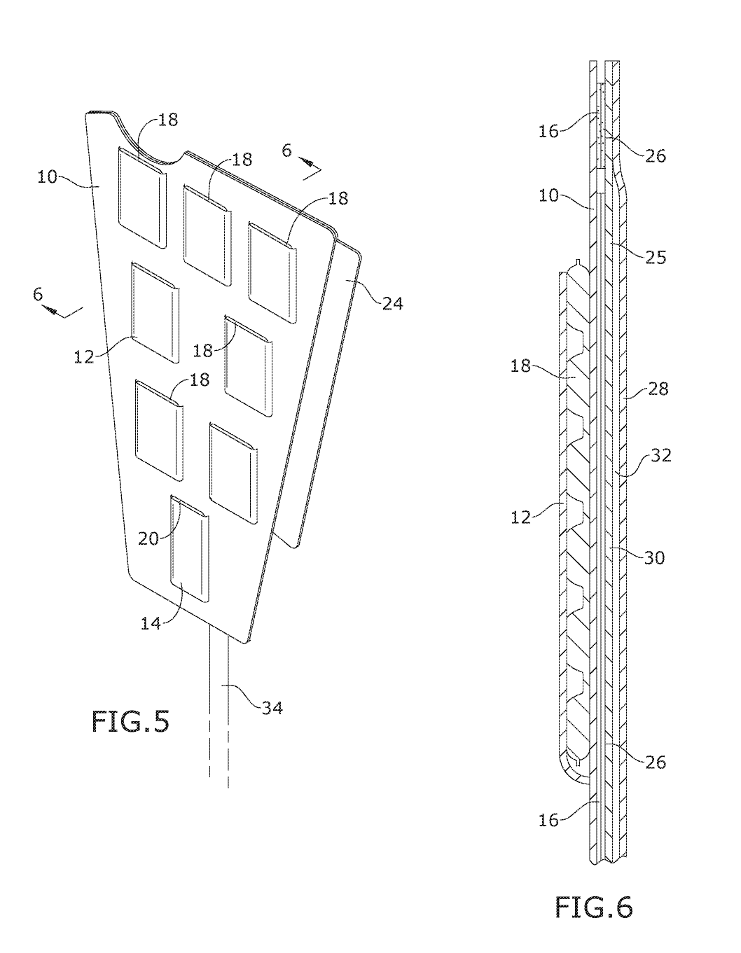

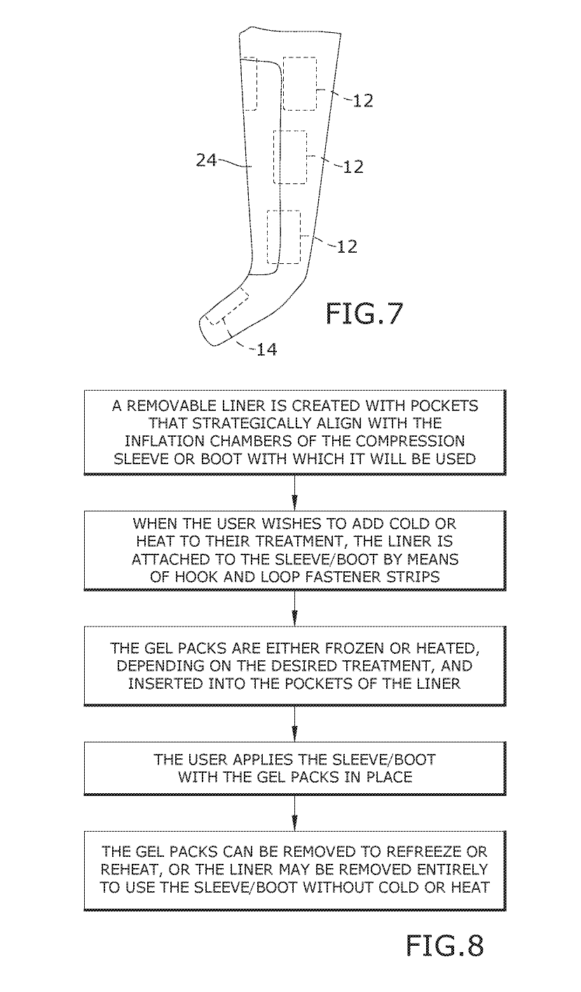

Referring to FIGS. 1 through 8, the present invention includes a therapeutic sleeve. The therapeutic sleeve includes a compression sleeve 25 having an inner sheet 30 and an outer sheet 28 made of flexible fluid impervious material sealingly fixed together to form at least one air pocket 32 therebetween. An inlet valve 34 is fluidly connected to the at least one air pocket 32 to fill the air pocket 32 with air via a pump. The present invention further includes a liner 10 including a sheet having an outer surface and an inner surface. At least one pocket 12 is coupled to the outer surface. A first connector 16, 26 couples the inner surface of the liner 10 to the inner sheet 30 of the compression sleeve 25. A second connector 24 is configured to removably couple the compression sleeve 25 to itself when wrapped around a limb of an animal, such as a human limb. The second connector 24 may include hook and loop fasteners, zippers, clips, and the like.

In certain embodiments, the liner 10 of the present invention may include a plurality of pockets 12. The pockets 12 removably retain a cold pack or a hot pack 18, depending on the type of therapy. Additionally, due to the number of pockets 12, a user may selectively treat certain areas by placing cold or hot packs in only pockets that are located to cover the injured or sore area of the limb. The liner 10 may also include a foot pocket 14 to removably secure foot hot and cold packs 20. The hot and cold packs 18, 20 may be gel packs.

The liner 10 may be fastened to the compression sleeve 25 or removably coupled to the compression sleeve 25. The term fastened means it is attached to the compression sleeve 25 and not easily removed without potentially damaging the compression sleeve 25. For example, fastened may include the liner 10 being stitched or adhered to the compression sleeve 25 by a strong glue. Alternatively, the liner 10 may be removably coupled to the compression sleeve 25. In such embodiments, the first connector 16, 26 may include one of hook strips and loop strips 16 fastened to the liner 10 and the other of the hook strips and the loop strips 26 fastened to the compression sleeve 25. The hook and loop strips 16, 26 removably retain the liner 10 to the compression sleeve 25.

A method of treating a limb of an animal may include the following steps: provide the compression sleeve 25 described above; provide the liner 10 described above; place at least one of a hot pack and a cold pack 18 in the at least one pocket 12; removably couple the inner surface of the liner 10 to the inner sheet 30 of the compression sleeve 25; wrap the compression sleeve 25 and the liner 10 around the limb of the animal so that the outer surface of the liner 10 is facing towards the limb; removably couple the compression sleeve 25 to itself, thereby securing the compression sleeve 25 and the liner to the limb; and pump air into the at least one air pocket 32 through the inlet valve 34. As mentioned above, the at least one pocket 12 may include a plurality of pockets 12. The method may further include placing hot packs or cold packs 18 in a portion of the plurality of pockets 12 for targeting an injured or sore area on the limb of the animal.

It should be understood, of course, that the foregoing relates to exemplary embodiments of the invention and that modifications may be made without departing from the spirit and scope of the invention as set forth in the following claims.

What is claimed is:

1. A therapeutic sleeve comprising:
a compression sleeve comprising an inner sheet and an outer sheet made of flexible fluid impervious material sealingly fixed together to form at least one air pocket therebetween and an inlet valve fluidly connected to the at least one air pocket;
a liner comprising a sheet having an outer surface and an inner surface, wherein at least one pocket is coupled to the outer surface;
a first connector configured to removably couple the inner surface of the liner to the inner sheet of the compression sleeve; and
a second connector configured to removably couple the compression sleeve to itself when wrapped around a limb of an animal.

2. The therapeutic sleeve of claim 1, further comprising at least one of a hot pack and a cold pack disposed within the at least one pocket.

3. The therapeutic sleeve of claim 2, wherein the hot pack or the cold pack are gel packs.

4. The therapeutic sleeve of claim 1, wherein the at least one pocket of the liner is a plurality of pockets coupled to the outer surface.

5. The therapeutic sleeve of claim 1, wherein the first connector comprises one of hook strips and loop strips fastened to the liner and the other of the hook strips and the loop strips fastened to the compression sleeve.

6. A method of treating a limb of an animal comprising:
providing a compression sleeve comprising an inner sheet and an outer sheet made of flexible fluid impervious material sealingly fixed together to form at least one air pocket therebetween and an inlet valve fluidly connected to the at least one air pocket;
providing a liner comprising a sheet having an outer surface and an inner surface, wherein at least one pocket is coupled to the outer surface;
placing at least one of a hot pack and a cold pack in the at least one pocket;
removably coupling the inner surface of the liner to the inner sheet of the compression sleeve;
wrapping the compression sleeve and the liner around the limb of the animal so that the outer surface of the liner is facing towards the limb;
removably coupling the compression sleeve to itself, thereby securing the compression sleeve and the liner to the limb; and
pumping air into the at least one air pocket through the inlet valve.

7. The method of claim 6, wherein the hot pack and the cold pack are gel packs.

8. The method of claim 6, wherein the at least one pocket of the liner is a plurality of pockets coupled to the outer surface.

9. The method of claim 6, wherein the liner is removably coupled to the compression by hook and loop fasteners.

10. A therapeutic sleeve comprising:
a compression sleeve comprising an inner sheet and an outer sheet of flexible fluid impervious material sealingly fixed together to form at least one air pocket therebetween and an inlet valve fluidly connected to the at least one air pocket;
a liner comprising a sheet having an outer surface and an inner surface, wherein at least one pocket is coupled to the outer surface;
at least one of a hot pack and a cold pack removably disposed within the at least one pocket;
a first connector fastening the inner surface of the liner to the inner sheet of the compression sleeve; and
a second connector configured to removably couple the compression sleeve to itself when wrapped around a limb of an animal.
